(19) United States
(12) Patent Application Publication
(10) Pub. No.: US 2015/0038361 A1
(43) Pub. Date: Feb. 5, 2015

(54) APPARATUS, METHODS, AND APPLICATIONS FOR POINT OF CARE MULTIPLEXED DIAGNOSTICS

(71) Applicant: Cornell University, Ithaca, NY (US)

(72) Inventors: David Erickson, Ithaca, NY (US); Matthew Mancuso, Bohemia, NY (US); Ethel Cesarman, Hoboken, NJ (US); Li Jiang, Ithaca, NY (US)

(21) Appl. No.: 14/377,962

(22) PCT Filed: Feb. 14, 2013
(86) PCT No.: PCT/US2013/026127

§ 371 Date: Aug. 11, 2014

Related U.S. Application Data
(60) Provisional application No. 61/598,599, filed on Feb. 14, 2012.
(60) Provisional application No. 61/751,020, filed on Jan. 10, 2013.

(51) Int. Cl. C12Q 1/70; G01N 33/569; G01N 33/553; C12Q 1/68; G01N 33/543

(52) U.S. Cl. CPC C12Q 1/705; C12Q 1/689; C12Q 1/6806; G01N 33/54313; G01N 33/553; G01N 33/56994; G01N 33/56911; C12Q 2600/112; C12Q 2600/158; C12Q 2600/16
USPC 506/9; 506/39

(57) ABSTRACT

Methods and systems for colorimetric detection of a target. Nucleic acid is obtained from a sample potentially containing two pathogens of interest, and is contacted with a plurality of nanoparticles. A first portion of the plurality of nanoparticles are functionalized with oligonucleotides complementary to a first region of the first target and oligonucleotides complementary to a second region of the first target, and a second portion of the plurality of nanoparticles are functionalized with oligonucleotides complementary to a first region of the second target and oligonucleotides complementary to a second region of the second target. The presence of the target nucleic acid causes a detectable colorimetric change, thereby diagnosing the presence of the pathogen.

### CROSS-REFERENCE TO RELATED APPLICATIONS

This application claims priority to U.S. Provisional Patent Application Ser. No. 61/598,599, filed on Feb. 14, 2012 and entitled “Apparatus, Methods, and Applications Pertaining to Point of Care Diagnostics,” and U.S. Provisional Patent Application Ser. No. 61/751,020, filed on Jan. 10, 2013 and entitled “Apparatus, Methods, and Applications Pertaining to Point of Care Diagnostics,” the entire disclosures of which are incorporated herein by reference.

### FIELD OF THE INVENTION

The present invention relates to methods, devices, and applications pertaining to point of care diagnostics and, more specifically, to multiplexed colorimetric point of care diagnostics.

### BACKGROUND

With the onset of the acquired immunodeficiency syndrome (“AIDS”) epidemic in the early 1980s, one of the first indicators of infected patients was the presence of red skin lesions, a symptom of a disease known as Kaposi's sarcoma (“KS”). Before the discovery of the cause of AIDS, KS and other opportunistic infections were often the first signs and biggest complications for infected individuals. During this time significant research efforts were made to determine the cause of AIDS, and in 1983 the human immunodeficiency virus (“HIV”) was discovered. A little over ten years later the cause of KS was first connected to a second virus, Kaposi's sarcoma-associated herpesvirus (“KSHV”), also given the alternative designation of human herpesvirus 8 (“HHV-8”).

KS remains the most prevalent cancer in untreated HIV-infected individuals, with some studies suggesting that it affects 1 in 20 HIV-positive patients. With the introduction of highly active anti-retroviral therapy (“HAART”), patients in the developed world have seen significant improvements in the management and treatment of KS, but it remains increased in incidence as compared to the pre-AIDS era in HIV-infected patients. Further, in many regions of the developing world such as Sub-Saharan Africa, both HIV and KS are endemic and KS is the fourth leading cancer in the region. In some countries such as Uganda, Kans. is the number one cause of cancer in men.

The infectious cause of KS is now well known to be the oncogenic herpesvirus KSHV or HHV-8. While the details of transmission are still being studied, it is most likely through saliva and in some regions KSHV rapidly spreads beginning in childhood affecting large portions of the population, reaching seroprevalence of over 50%. Like other herpesviruses, KSHV can establish a latent infection, and remains without causing any disease for the remaining life in most infected hosts, being necessary but not sufficient of KS development. In locations where the seroprevalence of KSHV is this high, the clinically relevant test is determining whether KSHV is present in a specific tumor, and not simply if it is present in a person's blood.

A second issue arises because of a number of other diseases have a similar presentation as Kaposi's sarcoma, and are part of the differential diagnosis. KS most often presents as a collection of red lesions, and when looked at on a typical hematoxylin and eosin stained histology slide has a number of unique features, including vascular spaces and proliferation of spindle cells thought to be of lymphatic endothelial origin. However, while these features are characteristic of KS, a number of other diseases, including bacillary angiomatosis (“BA”) caused by Bartonella henselae or quintana, and pyogenic granuloma with no known infectious cause, can often have a similar clinical and histological appearance and represent a diagnostic challenge.

In developed clinical settings, skin biopsies are easily processed for histology using advanced tools including tissue processing systems and microtomes. KS diagnosis can then be made after an H&E staining through microscopic evaluation by a pathologist, and when the histological characteristics are uncertain, the presence of KSHV is determined to confirm the diagnosis either with immunohistochemistry specific for unique KSHV proteins or PCR specific for unique KSHV DNA sequences. While the professional expertise and methods for sample preparation and diagnostic techniques are available in developed nations, they are scarce or nonexistent in many of the places where KS is most prevalent. If affordable point-of-care diagnostics could be created that are capable of distinguishing KS from other similar conditions, better treatment could be provided.

Ultimately, two unique challenges present themselves in the creation of point-of-care diagnostics for Kaposi's sarcoma in the developing world. The first is the requirement for the detection of KSHV in a biopsy sample without reliance on common laboratory technology. Extracting DNA from a skin biopsy sample using only simple, robust technology has thus far received little attention. The second challenge involves the presence of other diseases that can mimic KS, and thus the need for creating multiplexed detections that can distinguish one from the other. Further, these multiplexed detection systems need to be easily integrated to work with a small sample size, and in the presence of whatever surfactants and contamination is left over after DNA extraction.

While diagnostics in the developing world in general pose a number of challenges, some work has been done addressing them. Recently biosensors have been developing using mechanical, electrical, and optical techniques to detect bioanalytes in minute quantities. Yet for all of the successes of these biosensors, a number of limitations still exist, including the need to pre-process samples, the ability to work in a range of buffers (including those used to lyse cells), high sensitivity limits, and often a limited ability to detect multiple targets.

### BRIEF SUMMARY

It is therefore a principal object and advantage of to provide affordable, in-the-field point-of-care diagnostics.

It is another object and advantage of to provide methods, devices, and systems for extracting DNA from a skin biopsy sample using simple, robust technology.

It is a further object and advantage of to provide multiplexed detection methods, systems, and devices capable of distinguishing KS from other conditions.

Other objects and advantages of the present invention will in part be obvious, and in part appear hereinafter.

In accordance with the foregoing objects and advantages, a device for detecting the presence of a target in a sample, the device comprising: (i) an extraction chamber adapted to receive said sample and extract a first biomarker from said target if said target is present in said sample; (ii) a biomarker recognition element, wherein said biomarker recognition element is adapted to generate a first detectable signal in the presence of said first biomarker; (iii) a detection chamber in fluid communication with said extraction chamber, wherein said detection chamber is adapted to allow detection of said first detectable signal.

According to an aspect, the extraction chamber is at least a portion of a syringe-like apparatus, and comprises a lysis buffer.

According to an aspect, biomarker is a nucleic acid or a protein.

According to an aspect, the biomarker recognition element comprises a plurality of nanoparticles, said plurality of nanoparticles comprising: (i) a first plurality of nanoparticles functionalized with a biomarker recognition sequence complementary to a first region of said first biomarker; (ii) a second plurality of nanoparticles functionalized with a biomarker recognition sequence complementary to a second region of said first biomarker, wherein if said first target is present in said sample, said biomarker recognition sequence complementary to the first region of said biomarker and said biomarker recognition sequence complementary to a second region of said biomarker anneal to said extracted first biomarker and said first detectable signal is produced.

According to yet another aspect, the biomarker recognition element further comprises: (iii) a third plurality of nanoparticles functionalized with a biomarker recognition sequence complementary to a first region of a biomarker of a second target; and (iv) a fourth plurality of nanoparticles functionalized with a biomarker recognition sequence complementary to a second region of a biomarker of a second target, wherein if said second target is present in said sample, said biomarker recognition sequence complementary to the first region of said biomarker of said second target and said biomarker recognition sequence complementary to a second region of said biomarker of said second target anneal to the biomarker and a second detectable signal is produced.

According to an aspect, the sample is a biopsy.

According to another aspect, the detection chamber is adapted to concentrate said extracted biomarker, and can be a microfluidics chip

According to an aspect, the target is selected from the group consisting of Kaposi's sarcoma-associated herpesvirus, Bartonella quintana, Bartonella henselae, KSHV/HHV-8, EBV/HHV-4, CMV/HHV-1, HSV1/HHV-1, HSV2/HHV-2, HPV, HIV, Mycobacteria, Plasmodia falciparum, Plasmodia malariae, Chlamydia trachomatis, Neisseria gonorrhoeae, Bartonella bacteria, Vibrio cholera, dengue virus, and ebola virus.

According to an aspect, the device comprises a control element comprising: (i) a first plurality of nanoparticles functionalized with a control element recognition sequence complementary to a first region of a control element; and (ii) a second plurality of nanoparticles functionalized with a control element recognition sequence complementary to a second region of a control element, wherein if said second control element is present in said sample, said control element recognition sequence complementary to the first region of said control element and said control element recognition sequence complementary to a second region of said control element anneal to the control element and a detectable control signal is produced.

According to an aspect, a device for detecting the presence of a target in a biopsy, the device comprising: (i) an extraction chamber comprising a lysis buffer, wherein said extraction chamber is at least a portion of a syringe-like device adapted to receive said biopsy, and further wherein said extraction chamber is adapted to allow the extraction of a biomarker of said target from said biopsy if said target is present; and (ii) a plurality of nanoparticles, wherein said plurality of nanoparticles comprises: (a) a first plurality of nanoparticles functionalized with a biomarker recognition sequence complementary to a first region of said biomarker; (b) a second plurality of nanoparticles functionalized with a biomarker recognition sequence complementary to a second region of said biomarker, wherein if said first target is present in said biopsy, said biomarker recognition sequence complementary to the first region of said biomarker and said biomarker recognition sequence complementary to a second region of said biomarker anneal to said extracted biomarker and a first detectable signal is produced; (c) a third plurality of nanoparticles functionalized with a biomarker recognition sequence complementary to a first region of a biomarker of a second target; and (d) a fourth plurality of nanoparticles functionalized with a biomarker recognition sequence complementary to a second region of a biomarker of a second target, wherein if said second target is present in said sample, said biomarker recognition sequence complementary to the first region of said biomarker of said second target and said biomarker recognition sequence complementary to a second region of said biomarker of said second target anneal to the biomarker and a second detectable signal is produced; and (iii) a detection chamber in fluid communication with said extraction chamber, wherein said detection chamber is adapted to allow detection of said detectable signal.

According to an aspect, a method for detecting the presence of a target in a sample, the method comprising the steps of: (i) obtaining the sample; (ii) extracting a first biomarker from said sample if said target is present, wherein said biomarker is extracted in a syringe-like device adapted to receive said sample; (iii) contacting said first biomarker with a biomarker recognition element to generate a biomarker recognition mixture, wherein said biomarker recognition element is adapted to generate a first detectable signal in the presence of said first biomarker; (iv) transferring said biomarker recognition mixture to a detection chamber, wherein said detection chamber is in fluid communication with said syringe-like device; and (v) detecting said first detectable signal.

According to an aspect, the extracting step comprises the step of contacting said sample to a lysis buffer.

According to another aspect, the biomarker is a nucleic acid or a protein.

According to an aspect, the biomarker recognition element comprises a plurality of nanoparticles, said plurality of nanoparticles comprising: (i) a first plurality of nanoparticles functionalized with a biomarker recognition sequence complementary to a first region of said biomarker; (ii) a second plurality of nanoparticles functionalized with a biomarker recognition sequence complementary to a second region of said biomarker, wherein if said first target is present in said sample, said biomarker recognition sequence complementary to the first region of said biomarker and said biomarker recognition sequence complementary to a second region of said biomarker anneal to said extracted biomarker and a first detectable signal is produced.

According to another aspect, the biomarker recognition element further comprises: (iii) a third plurality of nanoparticles functionalized with a biomarker recognition sequence complementary to a first region of a biomarker of a second target; and (iv) a fourth plurality of nanoparticles functionalized with a biomarker recognition sequence complementary to a second region of a biomarker of a second target, wherein if said second target is present in said sample, said biomarker recognition sequence complementary to the first region of said biomarker of said second target and said biomarker recognition sequence complementary to a second region of said biomarker of said second target anneal to the biomarker and a second detectable signal is produced.

According to an aspect, the first detectable signal and second detectable signal are colorimetric signals.

According to another aspect, the method further comprises the step of amplifying said extracted first biomarker.

According to an aspect, a kit for detecting the presence of a target in a sample, said kit comprising: (i) a device comprising: (1) an extraction chamber adapted to receive said sample allow extraction of a biomarker of said target from said sample if said target is present; and (2) a detection chamber in fluid communication with said extraction chamber, wherein said detection chamber is adapted to allow detection of said detectable signal; and (ii) a biomarker recognition element, wherein said biomarker recognition element generates a first detectable signal in the presence of said first biomarker.

According to another aspect, the biomarker recognition element comprises a plurality of nanoparticles, said plurality of nanoparticles comprising: (i) a first plurality of nanoparticles functionalized with a biomarker recognition sequence complementary to a first region of said biomarker; (ii) a second plurality of nanoparticles functionalized with a biomarker recognition sequence complementary to a second region of said biomarker, wherein if said first target is present in said sample, said biomarker recognition sequence complementary to the first region of said biomarker and said biomarker recognition sequence complementary to a second region of said biomarker anneal to said extracted biomarker and a first detectable signal is produced.

According to an aspect, the plurality of nanoparticles further comprises: (iii) a third plurality of nanoparticles functionalized with a biomarker recognition sequence complementary to a first region of a biomarker of a second target; and (iv) a fourth plurality of nanoparticles functionalized with a biomarker recognition sequence complementary to a second region of a biomarker of a second target, wherein if said second target is present in said sample, said biomarker recognition sequence complementary to the first region of said biomarker of said second target and said biomarker recognition sequence complementary to a second region of said biomarker of said second target anneal to the biomarker and a second detectable signal is produced.

According to another aspect, the kit further comprises a control element, said control element comprising: (i) a first plurality of nanoparticles functionalized with a control element recognition sequence complementary to a first region of a control element; and (ii) a second plurality of nanoparticles functionalized with a control element recognition sequence complementary to a second region of a control element, wherein if said second control element is present in said sample, said control element recognition sequence complementary to the first region of said control element and said control element recognition sequence complementary to a second region of said control element anneal to the control element and a detectable control signal is produced.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a depiction of silver (a.) and gold (b.) nanoparticle aggregation and the corresponding change in absorbance, according to an embodiment;

FIG. 2 is a flowchart of a method for multiplex detection according to an embodiment;

FIG. 3 contains graphs depicting the absorbance of silver (a.) and gold (b.) nanoparticles functionalized with oligonucleotides according to an embodiment;

FIG. 4 is a melting temperature analysis according to an embodiment;

FIG. 5 is a graph of absorbance of BA-conjugated silver nanoparticles and KSHV-conjugated gold nanoparticles according to an embodiment;

FIG. 6 contains scanning electron micrographs of unaggregated (top) and aggregated (bottom) silver and gold nanoparticles functionalized with oligonucleotides according to an embodiment;

FIG. 7 contains graphs depicting the absorbance of silver and gold nanoparticles functionalized with oligonucleotides according to an embodiment;

FIG. 8 is a schematic of a sample processing and analysis device according to an embodiment; and

FIG. 9 is a graph of depicting the absorbance of metal nanoparticles functionalized with oligonucleotides according to an embodiment.

### DETAILED DESCRIPTION

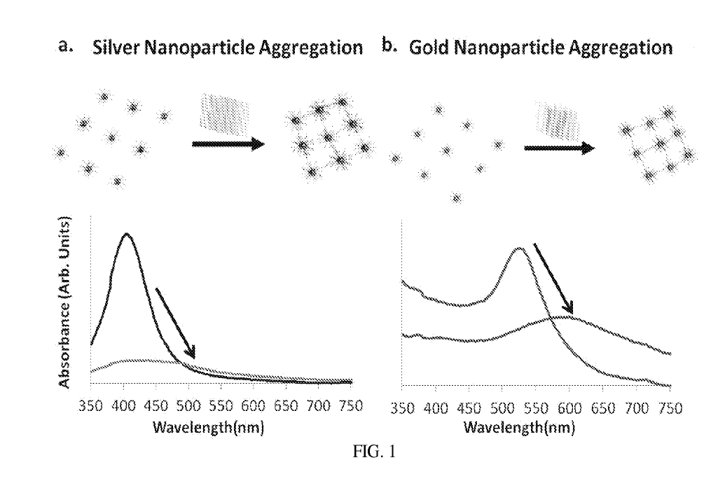

The invention may be more readily understood by reference to the following detailed description in connection with the accompanying figures and examples. Referring now to the drawings, wherein like reference numerals refer to like parts throughout, there is seen in FIG. 1 the use of surface plasmon resonance to create a colorimetric change in response to the presence of target nucleic acid. Silver and gold nanoparticles aggregate in the presence of target nucleic acid, and as the nanoparticles aggregate their surface plasmons couple, the resonance condition changes, and their characteristic optical peaks red shift. According to one embodiment, each type of nanoparticle can be functionalized to react differently, allowing for either color change reaction to take place independently. According to another embodiment, by using larger nanoparticles (such as, for example, approximately 50 nm) or a material with a higher optical cross section these colorimetric nanoparticle detection schemes can be optimized to reach sensitivities in the 50 pM to 1 nM range.

Described herein are, for example, single and multiplexed one-pot detection methods and systems that utilize surface plasmon resonance to create a colorimetric change in response to the presence of different target nucleic acids. For example, according to one embodiment, the single and multiplexed detection methods and systems can be used as a point-of-care diagnostic for detecting both KSHV nucleic acid and nucleic acid from a frequently confounding disease, bacillary angiomatosis. According to an embodiment, gold and silver nanoparticle aggregation reactions are tuned for each target and a multi-color change system is developed capable of detecting both targets down to, for example, levels between at least 1 nM and 2 nM. According to another embodiment, the methods and systems are integrated with microfluidic sample processing.

According to one embodiment, the method, kit, or device comprises a control element for qualitatively or quantitatively evaluating the detection reaction or process. For example, the control element can comprise a detection mechanism similar to a target detection mechanism described herein or known in the art. According to one embodiment, the control element comprises a first plurality of nanoparticles functionalized with a control element recognition sequence for a first region of the control element, and a second plurality of nanoparticles functionalized with a control element recognition sequence for a second region of a control element. If the control element is present in the sample, and if the detection reaction progresses under acceptable reaction conditions, the control element recognition sequence for the first region of the control element and the control element recognition sequence for the second region of the control element anneal to the control element and some detectable control signal is produced as described herein. If a reaction condition is not acceptable, or if there is another problem with the device, method, or reaction, then the detectable control signal is not produced and the user is alerted to an error in the device, method, or reaction that likely requires attention and/or correction. According to one embodiment, the control element is a stable and largely inert molecule that is added to the sample prior to processing. The control element can also quantifiably react with the control element recognition sequence to produce the detectable control signal under suitable conditions. The control element and control element recognition sequences could also be designed to produce different or modified detectable control signals depending on one or more conditions within the reaction, including temperature, UV exposure, and non-suitable lysis processing, among many others.

According to an embodiment, a sample such as a biopsy or other type of sample comprises a biomarker, the presence of which indicates the presence of the target. The biomarker could be the target, or the biomarker could be a component of the target. The biomarker can be, for example, a nucleic acid or a protein. According to another embodiment, the biomarker is a detectable viral component. According to yet another embodiment, the biomarker is a detectable molecule other than a nucleic acid or a protein, such as a mineral, an organic or inorganic polymer, a lipid or lipid metabolites, or another small molecule.

According to one embodiment, the presence of the target is detected using a nucleic acid—such as oligonucleotide or other nucleic acid—that is complementary to a nucleic acid sequence of the target. According to this embodiment, the detecting nucleic acid anneals to the target nucleic acid in order to facilitate detection. According to another embodiment, the presence of the target is detected using a non-nucleic acid target recognition component that is complementary to—and binds to and/or recognizes—a component of the target. For example, the target recognition component is a protein that recognizes and binds to a protein of the target. According to one embodiment, the target recognition component is an antibody or an antibody fragment that binds a target component such as a protein. Accordingly, the interaction between the target recognition component and the target or biomarker can occur via structural complementarity, nucleic acid-specific complementarity, and through other interacting mechanisms known in the art.

According to an embodiment, the presence of the target is detected using functionalized nanoparticles and colorimetric signals as described elsewhere herein. According to another embodiment, the presence of the target is detected using another detection mechanism, including but not limited to fluorescent or other dyes, PCR, wavelength absorption, enzyme-linked colorimetric detection, real-time fluorescence, and a wide variety of other detection methods known in the art.

Further, according to yet another embodiment, an extracted biomarker can be amplified prior to detection. For example, where the biomarker is a nucleic acid the method, kit, or device can be modified or designed to amplify the nucleic acid prior to a detection step. According to one embodiment, the extracted nucleic acid biomarker is amplified using PCR prior to downstream analysis. Other methods of amplifying the biomarker are known in the art.

Example Point-of-Care Diagnostic Methods

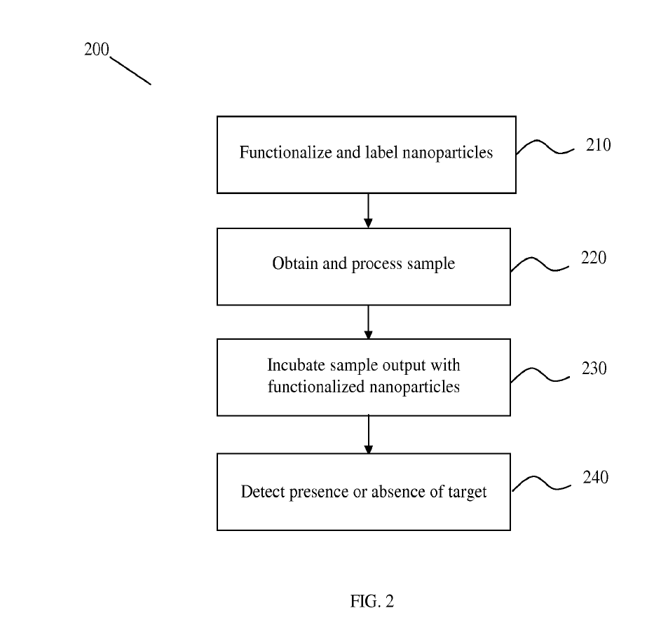

Reference is now made to FIG. 2 which illustrates a method 200, according to one embodiment, for multiplexed detection utilizing surface plasmon resonance to create a colorimetric change in response to the presence of different target nucleic acids. At step 210 of the example method shown in FIG. 2, nanoparticles are functionalized and labeled. According to one embodiment, the nanoparticles are a metal such as gold or silver and are functionalized using thiol-based chemistry, although other methods of functionalizing the nanoparticles are possible.

Once the nanoparticles are functionalized, they can be labeled with oligonucleotide sequences. At least a region of these oligonucleotide sequences are, according to a preferred embodiment, designed to be complementary to at least a region of nucleic acid of the target to be identified. For example, if the target to be detected is KSHV, the oligonucleotide sequence can be designed to target the nucleic acid that codes for the vCyclin protein, which is expressed during both the latent and lytic viral phases of KSHV. BLAST Primer Design is just one example method of designing suitable oligonucleotide sequences.

If two or more different oligonucleotide sequences are to be included in a single system designed for multiplex detection of two or more different targets, additional oligonucleotide sequences can be designed and added to nanoparticles. According to one embodiment, the genomes of the two or more different targets are compared to identify regions shared by all the targets, or, alternatively, unique to all the targets depending on the specific design of the system.

According to one embodiment, the oligonucleotides are modified by adding a polyadenine sequence to the 5′ end, followed by an alkyl thiol group used to bind the oligonucleotides to the nanoparticles. Other methods of attaching the oligonucleotide sequences to the nanoparticles are possible.

According to a preferred embodiment, a system designed to test for a single target comprises mixed or segregated populations of oligonucleotide functionalized nanoparticles. A first nanoparticle population comprises nanoparticles functionalized with a first oligonucleotide sequence (although note that there can be many, many copies of a sequence on a single nanoparticle) complementary to a first region of the target nucleic acid, and a second nanoparticle population comprises nanoparticles functionalized with a second oligonucleotide sequence complementary to a second region of the same target nucleic acid. Accordingly, when the target nucleic acid is introduced to the system, the first and second oligonucleotide sequences recognize and anneal to the target nucleic acid, thereby bringing together the first and second nanoparticle populations (as shown, for example, in FIG. 1).

According to another embodiment, a system designed to test for two targets comprises not only the first and second nanoparticle populations described in the previous paragraph, but further comprises a third nanoparticle population functionalized with a first oligonucleotide sequence (although note that there can be many, many copies of a sequence on a single nanoparticle) complementary to a first region of a second target nucleic acid, and a third nanoparticle population functionalized with a second oligonucleotide sequence complementary to a second region of the same, second target nucleic acid. Accordingly, this system comprises a mixture of four different functionalized nanoparticle populations. A system designed to test for three targets comprises not only the first, second, third, and fourth nanoparticle populations described in the previous paragraphs, but further comprises a fifth and sixth nanoparticle populations, and so on. An unlimited number of possible targets can be targeted and detected, so long as false positive detection events are designed around and minimized.

After the nanoparticles have been functionalized and labeled, or, alternatively, have been designed and commercially ordered, the nanoparticles are then ready for packaging and/or for use.

At step 220 of the example method shown in FIG. 2, a sample is obtained. The sample can be any tissue, fluid, or other component obtained directly from an individual, or can be a sample that has previously separated from or left behind by an individual (including, for example, a stool sample, a urine sample, or a saliva sample, among many others). Indeed, detection via the methods and systems described herein can be accomplished using, for example, tissue biopsies or body fluids including blood, saliva, sputum, urine and vaginal swabs. Non-biological samples can also be used to assess contamination, such as drinking water and food.

According to one embodiment, the sample is a skin biopsy. For example, the skin biopsy sample can be a sample obtained from a skin lesion, and can be, for example, a shave biopsy, punch biopsy, excisional biopsy, or an incisional biopsy.

The obtained sample is then processed to allow for target detection. Any method of processing the sample that allows for target detection, including for example, the release and/or isolation of nucleic acids from that sample, is suitable. According to one embodiment, described in greater detail elsewhere herein, the cells obtained in the sample are processed at the point of care using a lysis device designed to quickly and affordably input a sample and output nucleic acid from that sample for diagnosis and/or further downstream analysis.

At step 230 of the example method shown in FIG. 2, the output of the processed sample is incubated with the population(s) of oligonucleotide functionalized nanoparticles to allow the oligonucleotides to anneal to the target nucleic acid, if it is present. As described above, the target nucleic acid acts as a linker that allows the oligonucleotide functionalized nanoparticles to assemble. This nanoparticle assembly, which preferably occurs only in the presence of target nucleic acid, causes a shift in the nanoparticle surface plasmon resonance.

The shift in the nanoparticle surface plasmon resonance results in a detectable change in the solution comprising the oligonucleotide functionalized nanoparticle population(s), and at step 240 of the example method shown in FIG. 2, the presence or absence of the target nucleic acid—and, therefore, the presence or absence of the target—is determined by analyzing the incubated solution for the presence or absence of the detectable change. According to one embodiment, the detectable change is a visually-detectable variation in the color of the solution comprising the oligonucleotide functionalized nanoparticle population(s). The color variation may be such that it can be detected unaided by the human eye, or may be such that an optical device is required for detection. According to one embodiment, a diagnosis can be made from the presence or absence of the detectable change.

The multiplex detection methods, systems, and devices described herein can be utilized to detect a wide variety of viral, bacterial, or parasitic infectious agents. Specific organisms that can be detected with this methodology include, but are not limited to: KSHV/HHV-8, EBV/HHV-4, CMV/HHV-1, HS V1/HHV-1, HS V2/HHV-2, HPV (various strains), HIV, Mycobacteria (including MTB), Plasmodia (falciparum and malariae), Chlamydia trachomatis, Neisseria gonorrhoeae, Bartonella bacteria (cat scratch disease), Vibrio cholera, dengue virus and ebola virus, among many others.

According to one embodiment, the multiplex colorimetric detection methods, systems, and devices are utilized for the detection of HIV and syphilis. Although both diseases are treatable, in pregnant women they can be fatal to their children. Accordingly, a multiplexed colorimetric solution could provide a quick and affordable readout in a device that could provide the patient with more confidence in the result.

### Example 1

The following example describes a method, system, and device for point-of-care differential diagnosis of KS and BA based on a colorimetric one-pot gold and silver nanoparticle system. The multiplex system combines two oligonucleotide detection techniques in one solution, resulting in a system with two independent color change reactions depending on the target nucleic acid present in the sample. Importantly, the two oligonucleotide detection techniques in this multiplex system do not interfere with one other.

Primer Design and Selection

Oligonucleotide sequences were chosen for KSHV using BLAST Primer Design to determine short DNA sequences (˜20 base pairs) for DNA that codes for vCyclin, a KSHV protein known to express itself both during the latent and lytic viral phases. The fact that vCyclin is expressed both latently and lytically could later be useful, because direct detection of extracted RNA could provide an additional template for amplification. Bacillary angiomatosis, a bacterial infection, can be caused by two different species, Bartonella quintana and henselae, and primers were designed to be specific to both agents. Briefly, the two bacteria genomes were compared to find conserved regions, a reference genome was created out of the conserved regions, and BLAST Primer Design was used to find oligonucleotides specific to these regions. A 15 base long polyadenine sequence was added to the 5′ end of the sequences, followed by an alkyl thiol group used to bind the oligonucleotides to gold particles. All oligonucleotides were ordered from Invitrogen® (Grand Island, N.Y.), and their sequence information can be found in TABLE 1.

TABLE 1
Probe and Target Sequences for KSHV and BA

| | | T_{M} (° C.) |
|---|---|---|
| | | 300 mM |
| Name | Sequence | NaCl |
| KSHV Probe 1 | AAAAAAAAAAAAAAAGCCAACGTCATTCCGCAGGA | 76.1 |
| | T | |
| KSHV Probe 2 | AAAAAAAAAAAAAAAAGGCTGTGCGCTGTTGGTTC | 78.7 |
| | CT | |
| KSHV Target | ATCCTGCGGAATGACGTTGGCAGGAACCAACAGCG | 96.5 |
| | CACAGCCT | |
| Bartonella Probe 1 | AAAAAAAAAAAAAAACCAATCGGTGGAGACGG | 70.2 |
| Bartonella Probe 2 | AAAAAAAAAAAAAAACGCTGACCAAGAGCAGGA | 71.3 |
| Bartonella Target | CCGTCTCCACCGATTGGTCCTGCTCTTGGTCAGCG | 94.2 |
| Sequence | | |

Gold and Silver Nanoparticle Functionalization

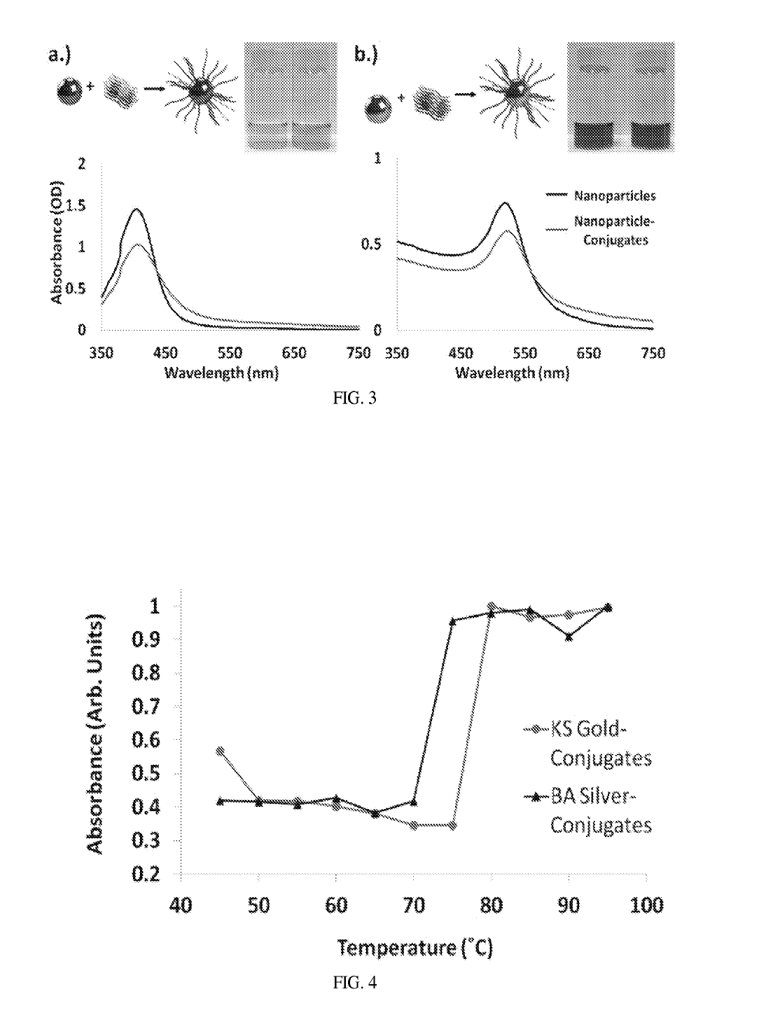

Gold and silver particles with average diameters of 15 and 20 nm, respectively, were functionalized using thiol based chemistry. These sizes were chosen as a compromise between larger particles which generally provide higher sensitivity, and smaller particles which are generally easier to make stable in salt solutions. Briefly, 50 μL of 100 μM oligonucleotides with 5′ alkyl thiol groups was added to 1 mL solutions of gold (3 nM) and silver (750 pM) nanoparticles in excess and allowed to react overnight. The solution was then brought to 10 mM sodium phosphate and 0.01% sodium dodecyl sulfate (SDS), and again given 24 hr to react. This process was repeated, this time adding sodium chloride, resulting in final concentrations of 100 mM, 200 mM, and 300 mM, each time with 24 hours in between. These increasing molarity salt solutions are used to screen electrostatic interactions between DNA strands, ultimately allowing for a higher density layer to be formed on the surface of the nanoparticles. After the final incubation period, solutions were spun down and resuspended in 0.01% SDS three times to remove excess oligonucleotides, and finally brought to 10 mM Sodium Phosphate and 300 mM Sodium chloride. FIG. 3 depicts silver (a) and gold (b) nanoparticles functionalized using thiolated oligonucleotides. After conjugation, both spectrums red shifted by approximately 1-3 nm.

Melting Temperature Analysis

Gold and silver nanoparticle-based aggregation can have specificity high enough to determine single nucleotide mismatches between targets. A perfect and one nucleotide mismatched target have different melting temperatures, and by measuring what temperature the nanoparticles disassociate one can distinguish between the two. A similar disassociation temperature is determined here for a correct target for both nanoparticle systems, and further detection reactions are performed at a temperature just below this threshold to insure incorrect targets don't cause any aggregation. KSHV and Bartonella DNA (10 nM) sequences were added to solutions of conjugated gold and silver nanoparticles respectively, and the solutions were allowed 4 hr to aggregate. Then, the solutions were heated in 5 degree increments from 45° C. to 95° C. to determine at what temperature the nanoparticles disassociated.

KSHV and BA Detection and Sensitivity Measurements

Solutions of gold and silver nanoparticles were mixed to yield a final concentration of 1.5 nM gold nanoparticles and 325 pM silver nanoparticles. Due to silver's higher absorption cross section, a lower concentration was used. Target and Control DNA were added at concentrations of 5 nM and solutions were kept 2 hours at 65° C. to react before their absorbance measurements were recorded. Similarly, experiments were conducted to measure the limit of detection of the system. Different concentrations of DNA from 10 pM to 1 uM were added to 40 μL of both silver and gold independently to measure the sensitivity of each channel. Solutions were given 2 hr at 65° C. to react, and their near UV and visible spectrums were collected.

Materials and Instrumentation

Spectrophotometric measurements were taken using a Spectramax® plus 384 (Molecular Devices, Sunnyvale, Calif.) in the Nanobiotechnology Center at Cornell University. SEMs were taken on a Zeiss® Ultra (Oberkochen, Germany) in the Cornell Center for Nanofabrication. Gold nanoparticles were purchased from Nanopartz (Loveland, Colo.). All other reagents were purchased from Sigma-Aldrich (St. Louis, Mo.).

Nanoparticle-Oligonucleotide Conjugation

The attachment of oligonucleotides to gold and silver nanoparticles yielded homogenous stable solutions of nanoparticle conjugates the same color as the original solution. As in previous work, a small change of roughly 1 to 3 nm in nanoparticle resonance was observed in accordance with the nanoparticle conjugations, as shown in FIG. 3. Decreases in absorbance were also observed due to incomplete collection of nanoparticles during excess oligonucleotide removal. The final gold nanoparticle solutions were stable for greater than 1 month at room temperature, while the silver particles were stable for approximately two weeks. This difference in stability is likely attributed to the different reaction constants between gold and thiol and silver and thiol.

Melting Temperature Analysis

The results of the melting temperature analysis indicated that the KSHV functionalized nanoparticles disassociated between 75° C. and 80° C., and the Bartonella functionalized nanoparticles between 70° C. and 75° C., as shown in FIG. 4. These results line up well with the expected melting temperatures of the oligonucleotide probes, which can be found in TABLE 1. Further, the lower melting temperature of the Bartonella probes agrees well with the length of the probes being 5 nucleotides shorter. These temperature results were used to choose 65° C. as the temperature that the detection and sensitivity experiments were conducted at to prevent nonspecific aggregation.

Multiplexed KSHV and BA DNA Detection Experiments

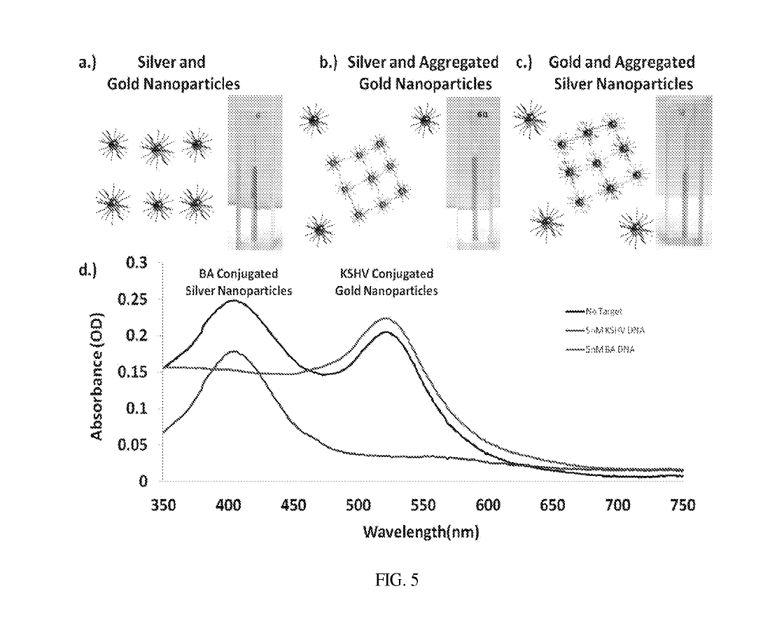

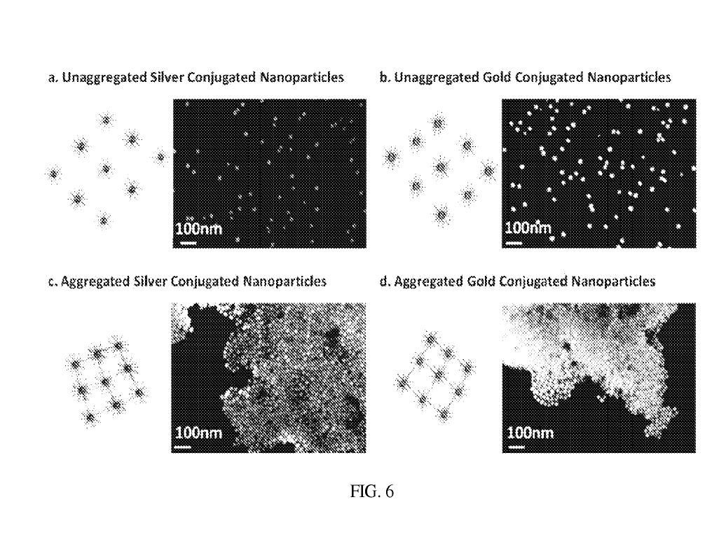

In experiments using both KSHV functionalized gold nanoparticles and Bartonella functionalized silver nanoparticles (FIG. 5a), upon successful aggregation and detection of one target, the multiplexed solution displayed a color more similar to the non-aggregated solution. For example, when Bartonella target DNA (BA DNA) was introduced to the solution the silver nanoparticles aggregated and the solution turned to a pink color, more dependent on the surface plasmon characteristics of the unaggregated gold particles (FIG. 5b). When KS DNA was introduced the gold nanoparticles aggregated and the solution changed to a murky yellow-orange color, more dependent on the silver nanoparticles (FIG. 5c). Spectrophotometric analysis also revealed that only the wavelength resonant peak of the nanoparticle aggregate was affected by the detection of a single target (FIG. 5d). A small change in the absorption at the non-target-corresponding resonant wavelength is observed due to a change in the corresponding resonant peak's tail, but the resonant peaks wavelength did not change. Further, scanning electron micrographs reveal that upon introduction of a target, an aggregation reaction does indeed occur (FIG. 6). For the gold nanoparticles a color change could be visually observed as early as 30 minutes to 1 hour after addition of DNA and for the silver nanoparticles as early as one hour after the addition of target. Measuring the absorbance of the solutions changes were observed as soon as 10 to 20 minutes after the addition of target.

When gold nanoparticles and silver nanoparticles were mixed and stored together the silver nanoparticles would gradually and nonspecifically aggregate over the course of two to three days, even in the presence of no target. Presumably this aggregation was caused by a reaction between the thiol groups of the Bartonella probe DNA attached to the silver nanoparticles reacting with the gold nanoparticles because of thiol and gold's greater reaction constant. However, over the time span of our reactions, no change was observed in the silver nanoparticles, allowing for multiplexed detection in one solution.

Sensitivity Experiments

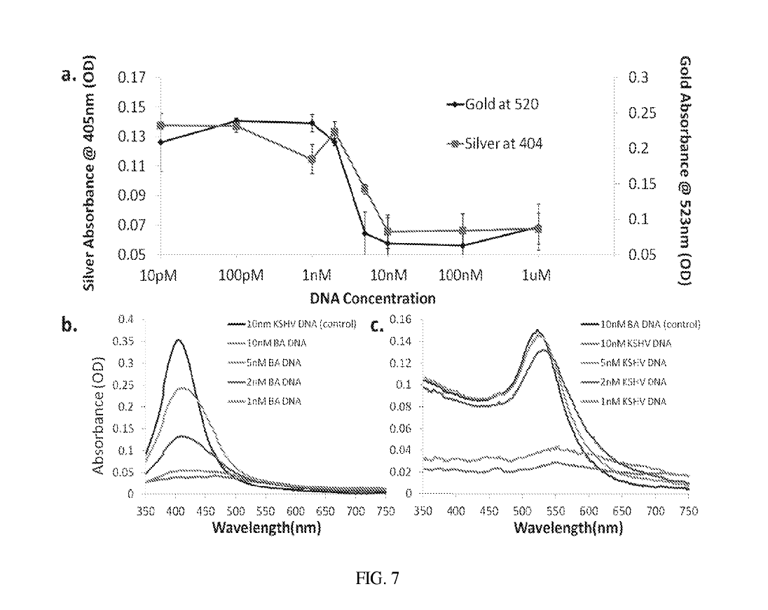

Detection reactions were carried out at various target DNA concentrations to determine the limit of detection of the system. The results indicates that the limit of detection of the gold nanoparticles is approximately 2 nM, and for the silver nanoparticles is approximately 1 nM (FIG. 7). The limit of detection of the silver nanoparticles is likely higher because their higher absorption cross section allows for a lower concentration of nanoparticles that can aggregate in the presence of less DNA. These results line up well with previous nanoparticle-based colorimetric detection, which shows limits of detection for gold around approximately 1 nM, and for silver around approximately 100 pM.

While these limits of detection are high for the detection of unamplified DNA, there are a number of techniques that can be implemented to allow a multiplexed method or system to directly detect extracted DNA. One example previously mentioned involves evanescently coupling light from illuminated glass slides into the nanoparticles to excite them as opposed to a broadband source. A limit of detection of 300 fM has been reported, demonstrating how this simple light source can provide an almost 1000 fold increase in sensitivity using the same nanoparticles. Ultimately, this system was used to detect unamplified genomic DNA. A second technique which could be used to directly detect KSHVs presence involves detecting mRNA already transcribed from the genomic DNA. As explained previously, vCyclin is expressed both latently and lytically, and orders of magnitude more copies could be available for detection.

Although this example demonstrates multiplexed detection using two targets, according to another embodiment nanoparticles of other shapes, sizes, and materials are utilized to design a multiplexed solution capable of many colorimetric detection reactions for different targets. In addition to nanospheres like those described above, nano-rods, prisms, bipyramids, and a number of other geometries exist with different SPR wavelengths. Depending on how much overlap is allowed between SPR peaks of different nanoparticles, anywhere from a handful to dozens of detections could be carried out within the width of the visible spectrum.

### Example 2

### Sample Processing

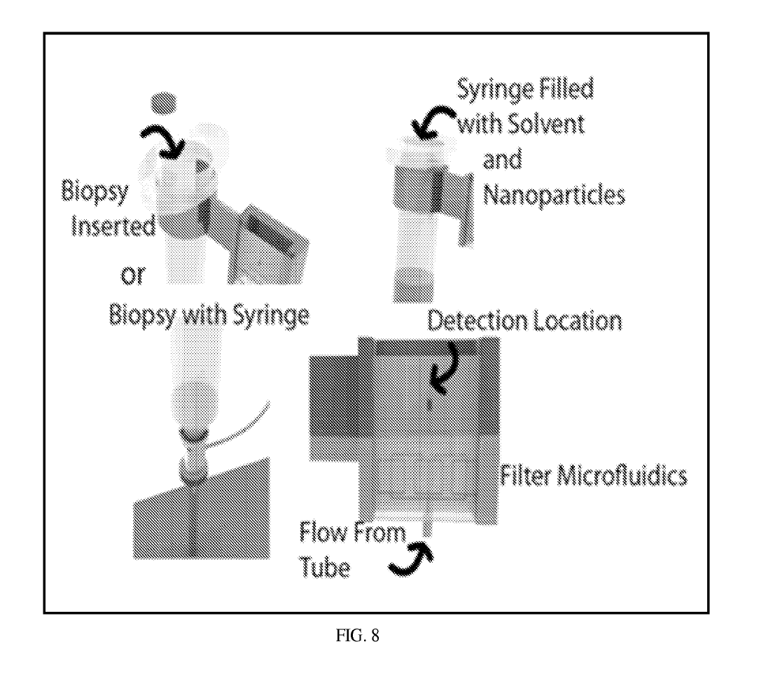

Methods, systems, and devices for sample processing according to an embodiment. To perform detection of target nucleic acid from a sample at the point of care, it is preferable to have an affordable and easily portable device not only for detection, but also for processing of obtained samples. According to one embodiment is provided a generally handheld device used for sample processing, nanoparticle incubation, and detection. For example, according to one embodiment the device could be a syringe, similar to the device depicted in FIG. 8. The device could similarly be a syringe-like device generally comprising a housing and a plunger or pushing means for forceably moving components from one area of the housing, device, or kit to another area of the housing, device, or kit.

According to an embodiment, the processing device comprises an input for the sample, such as a skin biopsy. The sample is then processed by surfactant and/or proteases which lyse the cells within the sample and/or degrade the proteins in the sample while yielding as much nucleic acid from the sample as possible for downstream detection. This step, and any of the following steps, can occur very rapidly (seconds or less), or can require a matter of minutes or hours for completion.

Once the nucleic acid is obtained and/or isolated from the cells within the sample, that nucleic acid can be incubated with the nanoparticle population(s) also located within the device. According to an embodiment, the nanoparticle population(s) are stored in a portion of the device separate from the lysis portion of the device. Gravity, manual or automatic force, or other methods of moving the sample and/or obtained nucleic acid from one portion or section of the device to another can be utilized.

Once the incubation step is complete, the device can be examined for the detection event signaling the presence of the target nucleic acid in the sample. If no detection event is observed, it is hypothesized that no target nucleic acid, or an undetectable level of the target, was present in the sample. If a detection event is observed, it is hypothesized that the target nucleic acid is indeed present in the sample.

According to one example, a cell pellet (a pseudo-biopsy) containing KSHV was added to the lysis syringe with a lysis buffer. For example, there are a number of different possible surfactants that could be used in the lysis buffer, including but not limited to SDS. After an incubation of approximately 20 minutes, the solution was put through a spin column to remove the lysis buffer and any non-DNA materials. PCR using KSHV genome specific primers and gel electrophoresis were then performed, and a PCR product was observed, thereby revealing the presence of KSHV in the sample. In a preferred embodiment, the spin column step is replaced with another mechanism for removing or neutralizing the lysis buffer and/or removing non-DNA materials.

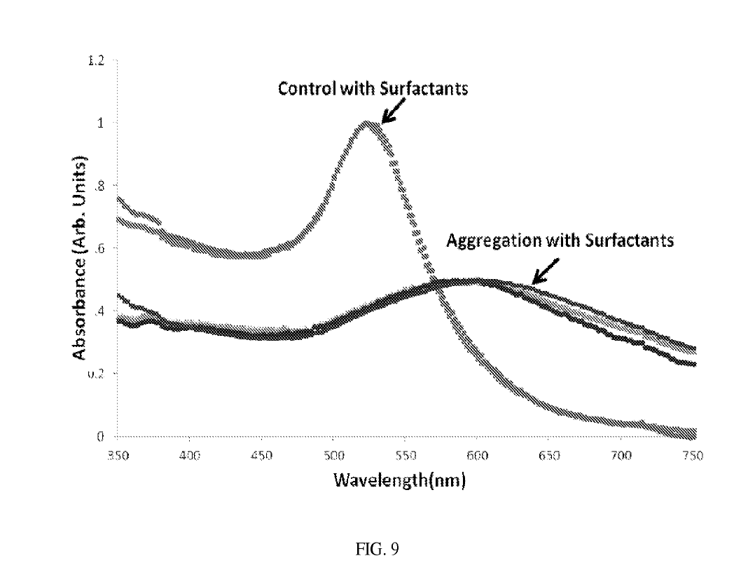

According to another example, a gold nanoparticle aggregation reaction as described above was allowed to progress in the presence of either SDS, Triton X-100, or Tween-20. A total of 10 nM KSHV target DNA was added to the oligonucleotide nanoparticle populations, and with each of the surfactants the nanoparticles aggregated and underwent a color change reaction as shown in FIG. 9. This suggests that the detection reaction functions properly downstream of the lysis step without an extra step for filtration or separation.

### Example 3

### Microfluidic Concentration

Methods, systems, and devices for microfluidic concentration according to an embodiment. According to an embodiment, a passive microfluidic device such as a microfluidic chip is utilized to concentrate the nanoparticle solution into a smaller volume, thereby improving the detection event (e.g., the color change) without requiring more target nucleic acid. Currently, a potentially limiting factor for observing the detection event in the presence of target nucleic acid is that there must be enough nanoparticles present to produce a visible color change, and that there must be enough target nucleic acid to link together a sufficient number of nanoparticles in order to produce a visible color change. According to one embodiment, the nanoparticles are concentrated using a microfluidic device, thereby improving the color change. The lower limit detection of the system could therefore be modified by using nanoparticles at concentrations that are not visible to bind to target DNA, and then concentrating any resulting aggregates using a microfluidic device.

A number of other possible solutions exist for increasing the sensitivity of colorimetric nanoparticle-based detection, including detecting amplified RNA targets such as vCyclin RNA, or using evanescent coupling into the particles to measure only scattered light.

Although the invention may be described in connection with a preferred embodiment, it should be understood that modifications, alterations, and additions can be made to the invention without departing from the scope of the invention as defined by the claims.

What is claimed is:

1. A device for detecting the presence of a target in a sample, the device comprising:
an extraction chamber adapted to receive said sample and extract a first biomarker from said target if said target is present in said sample;
a biomarker recognition element, wherein said biomarker recognition element is adapted to generate a first detectable signal in the presence of said first biomarker;
a detection chamber in fluid communication with said extraction chamber, wherein said detection chamber is adapted to allow detection of said first detectable signal.

2. The device of claim 1, wherein said extraction chamber is at least a portion of a syringe-like apparatus.

3. The device of claim 1, wherein said extraction chamber comprises a lysis buffer.

4. The device of claim 1, wherein said biomarker is a nucleic acid.

5. The device of claim 1, wherein said biomarker is a protein.

6. The device of claim 1, wherein said biomarker recognition element comprises a plurality of nanoparticles, said plurality of nanoparticles comprising: (i) a first plurality of nanoparticles functionalized with a biomarker recognition sequence complementary to a first region of said first biomarker; (ii) a second plurality of nanoparticles functionalized with a biomarker recognition sequence complementary to a second region of said first biomarker, wherein if said first target is present in said sample, said biomarker recognition sequence complementary to the first region of said biomarker and said biomarker recognition sequence complementary to a second region of said biomarker anneal to said extracted first biomarker and said first detectable signal is produced.

7. The device of claim 6, wherein said biomarker recognition element further comprises: (iii) a third plurality of nanoparticles functionalized with a biomarker recognition sequence complementary to a first region of a biomarker of a second target; and (iv) a fourth plurality of nanoparticles functionalized with a biomarker recognition sequence complementary to a second region of a biomarker of a second target, wherein if said second target is present in said sample, said biomarker recognition sequence complementary to the first region of said biomarker of said second target and said biomarker recognition sequence complementary to a second region of said biomarker of said second target anneal to the biomarker and a second detectable signal is produced.

8. The device of claim 1, wherein said sample is a biopsy.

9. The device of claim 1, wherein said detection chamber is adapted to concentrate said extracted biomarker.

10. The device of claim 9, wherein said detection chamber is a microfluidics chip

11. The device of claim 1, wherein said target is selected from the group consisting of Kaposi's sarcoma-associated herpesvirus, Bartonella quintana, Bartonella henselae, KSHV/HHV-8, EBV/HHV-4, CMV/HHV-1, HSV1/HHV-1, HSV2/HHV-2, HPV, HIV, Mycobacteria, Plasmodia falciparum, Plasmodia malariae, Chlamydia trachomatis, Neisseria gonorrhoeae, Bartonella bacteria, Vibrio cholera, dengue virus, and ebola virus.

12. The device of claim 1, further comprising:
a control element, said control element comprising: (i) a first plurality of nanoparticles functionalized with a control element recognition sequence complementary to a first region of a control element; and (ii) a second plurality of nanoparticles functionalized with a control element recognition sequence complementary to a second region of a control element, wherein if said second control element is present in said sample, said control element recognition sequence complementary to the first region of said control element and said control element recognition sequence complementary to a second region of said control element anneal to the control element and a detectable control signal is produced.

13. A device for detecting the presence of a target in a biopsy, the device comprising:
an extraction chamber comprising a lysis buffer, wherein said extraction chamber is at least a portion of a syringe-like device adapted to receive said biopsy, and further wherein said extraction chamber is adapted to allow the extraction of a biomarker of said target from said biopsy if said target is present; and
a plurality of nanoparticles, wherein said plurality of nanoparticles comprises: (i) a first plurality of nanoparticles functionalized with a biomarker recognition sequence complementary to a first region of said biomarker; (ii) a second plurality of nanoparticles functionalized with a biomarker recognition sequence complementary to a second region of said biomarker, wherein if said first target is present in said biopsy, said biomarker recognition sequence complementary to the first region of said biomarker and said biomarker recognition sequence complementary to a second region of said biomarker anneal to said extracted biomarker and a first detectable signal is produced; (iii) a third plurality of nanoparticles functionalized with a biomarker recognition sequence complementary to a first region of a biomarker of a second target; and (iv) a fourth plurality of nanoparticles functionalized with a biomarker recognition sequence complementary to a second region of a biomarker of a second target, wherein if said second target is present in said sample, said biomarker recognition sequence complementary to the first region of said biomarker of said second target and said biomarker recognition sequence complementary to a second region of said biomarker of said second target anneal to the biomarker and a second detectable signal is produced; and
a detection chamber in fluid communication with said extraction chamber, wherein said detection chamber is adapted to allow detection of said detectable signal.

14. A method for detecting the presence of a target in a sample, the method comprising the steps of:
obtaining the sample;
extracting a first biomarker from said sample if said target is present, wherein said biomarker is extracted in a syringe-like device adapted to receive said sample;
contacting said first biomarker with a biomarker recognition element to generate a biomarker recognition mixture, wherein said biomarker recognition element is adapted to generate a first detectable signal in the presence of said first biomarker;
transferring said biomarker recognition mixture to a detection chamber, wherein said detection chamber is in fluid communication with said syringe-like device; and
detecting said first detectable signal.

15. The method of claim 14, wherein said extracting step comprises the step of contacting said sample to a lysis buffer.

16. The method of claim 14, wherein said biomarker is a nucleic acid.

17. The method of claim 14, wherein said biomarker is a protein.

18. The method of claim 14, wherein biomarker recognition element comprises a plurality of nanoparticles, said plurality of nanoparticles comprising: (i) a first plurality of nanoparticles functionalized with a biomarker recognition sequence complementary to a first region of said biomarker; (ii) a second plurality of nanoparticles functionalized with a biomarker recognition sequence complementary to a second region of said biomarker, wherein if said first target is present in said sample, said biomarker recognition sequence complementary to the first region of said biomarker and said biomarker recognition sequence complementary to a second region of said biomarker anneal to said extracted biomarker and a first detectable signal is produced.

19. The method of claim 18, wherein biomarker recognition element further comprises: (iii) a third plurality of nanoparticles functionalized with a biomarker recognition sequence complementary to a first region of a biomarker of a second target; and (iv) a fourth plurality of nanoparticles functionalized with a biomarker recognition sequence complementary to a second region of a biomarker of a second target, wherein if said second target is present in said sample, said biomarker recognition sequence complementary to the first region of said biomarker of said second target and said biomarker recognition sequence complementary to a second region of said biomarker of said second target anneal to the biomarker and a second detectable signal is produced.

20. The method of claim 14, wherein said target is selected from the group consisting of Kaposi's sarcoma-associated herpesvirus, Bartonella quintana, Bartonella henselae, KSHV/HHV-8, EBV/HHV-4, CMV/HHV-1, HSV1/HHV-1, HSV2/HHV-2, HPV, HIV, Mycobacteria, Plasmodia falciparum, Plasmodia malariae, Chlamydia trachomatis, Neisseria gonorrhoeae, Bartonella bacteria, Vibrio cholera, dengue virus, and ebola virus.

21. The method of claim 14, said first detectable signal and said second detectable signals are colorimetric signals.

22. The method of claim 14, wherein said sample is a biopsy.

23. The method of claim 14, further comprising the step of concentrating said plurality of nanoparticles after said contacting step and before said detecting steps.

24. The method of claim 14, further comprising the step of amplifying said extracted first biomarker.

25. A kit for detecting the presence of a target in a sample, said kit comprising:
a device comprising: (i) an extraction chamber adapted to receive said sample allow extraction of a biomarker of said target from said sample if said target is present; and (ii) a detection chamber in fluid communication with said extraction chamber, wherein said detection chamber is adapted to allow detection of said detectable signal; and
a biomarker recognition element, wherein said biomarker recognition element generates a first detectable signal in the presence of said first biomarker.

26. The kit of claim 25, wherein said biomarker recognition element comprises a plurality of nanoparticles, said plurality of nanoparticles comprising: (i) a first plurality of nanoparticles functionalized with a biomarker recognition sequence complementary to a first region of said biomarker; (ii) a second plurality of nanoparticles functionalized with a biomarker recognition sequence complementary to a second region of said biomarker, wherein if said first target is present in said sample, said biomarker recognition sequence complementary to the first region of said biomarker and said biomarker recognition sequence complementary to a second region of said biomarker anneal to said extracted biomarker and a first detectable signal is produced.

27. The kit of claim 26, wherein said plurality of nanoparticles further comprises: (iii) a third plurality of nanoparticles functionalized with a biomarker recognition sequence complementary to a first region of a biomarker of a second target; and (iv) a fourth plurality of nanoparticles functionalized with a biomarker recognition sequence complementary to a second region of a biomarker of a second target, wherein if said second target is present in said sample, said biomarker recognition sequence complementary to the first region of said biomarker of said second target and said biomarker recognition sequence complementary to a second region of said biomarker of said second target anneal to the biomarker and a second detectable signal is produced.

28. The kit of claim 25, further comprising:
a control element, said control element comprising: (i) a first plurality of nanoparticles functionalized with a control element recognition sequence complementary to a first region of a control element; and (ii) a second plurality of nanoparticles functionalized with a control element recognition sequence complementary to a second region of a control element, wherein if said second control element is present in said sample, said control element recognition sequence complementary to the first region of said control element and said control element recognition sequence complementary to a second region of said control element anneal to the control element and a detectable control signal is produced.

29. The kit of claim 25, wherein said sample is a biopsy.
